(19) United States
(12) Patent Application Publication
(10) Pub. No.: US 2010/0109662 A1
(43) Pub. Date: May 6, 2010

(54) ARRANGEMENT AND METHOD FOR INFLUENCING AND/OR DETECTING MAGNETIC PARTICLES IN A REGION OF ACTION

(75) Inventors: Bernhard Gleich, Eindhoven (NL); Jurgen Weizenecker, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/519,774

(22) PCT Filed: Dec. 14, 2007
(86) PCT No.: PCT/IB2007/055134

§ 371 Date: Nov. 11, 2009

(30) Foreign Application Priority Data
Dec. 20, 2006 (EP) 06126575.7

(51) Int. Cl. G01R 33/00

(52) U.S. Cl. 324/260

(57) ABSTRACT

An arrangement and a method for influencing and/or detecting magnetic particles in a region of action is disclosed, which arrangement comprises: selection means for generating a magnetic selection field having a pattern in space of its magnetic field strength such that a first sub-zone having a low magnetic field strength and a second sub-zone having a higher magnetic field strength are formed in the region of action, drive means for changing the position in space of the two sub-zones in the region of action by means of a magnetic drive field so that the magnetization of the magnetic particles changes locally, wherein the selection means comprise at least one permanent magnet comprising a high resistive permanent magnet material.

The present invention relates to an arrangement for influencing and/or detecting magnetic particles in a region of action. Furthermore, the invention relates to a method for influencing and/or detecting magnetic particles in a region of action.

The arrangement and the method of this kind is known from German patent application DE 101 51 778 A1. In the case of the method described in that publication, first of all a magnetic field having a spatial distribution of the magnetic field strength is generated such that a first sub-zone having a relatively low magnetic field strength and a second sub-zone having a relatively high magnetic field strength are formed in the examination zone. The position in space of the sub-zones in the examination zone is then shifted, so that the magnetization of the particles in the examination zone changes locally. Signals are recorded which are dependent on the magnetization in the examination zone, which magnetization has been influenced by the shift in the position in space of the sub-zones, and information concerning the spatial distribution of the magnetic particles in the examination zone is extracted from these signals, so that an image of the examination zone can be formed. Such an arrangement and such a method have the advantage that it can be used to examine arbitrary examination objects—e.g. human bodies—in a non-destructive manner and without causing any damage and with a high spatial resolution, both close to the surface and remote from the surface of the examination object.

Known arrangements of this type have shown the disadvantage that coils or permanent magnets used to generate a non-uniform magnetic field need to produce very strong fields—especially strong gradient fields—in order to enable the arrangements to realize a given spatial resolution, the strong fields leading in general to high complexity of the arrangements, to a comparably high energy consumption, high cooling efforts, and an expensive overall setup of the known arrangements.

It is therefore an object of the present invention to provide an arrangement and a method of the kind mentioned initially, where the spatial resolution can be enhanced without the negative side effects mentioned.

The above object is achieved by an arrangement for influencing and/or detecting magnetic particles in a region of action, wherein the arrangement comprises selection means for generating a magnetic selection field having a pattern in space of its magnetic field strength such that a first sub-zone having a low magnetic field strength and a second sub-zone having a higher magnetic field strength are formed in the region of action, drive means for changing the position in space of the two sub-zones in the region of action by means of a magnetic drive field so that the magnetization of the magnetic particles changes locally, wherein the selection means comprise at least one permanent magnet comprising a high resistive permanent magnet material.

The inventive arrangement according to the present invention has the advantage that it is possible to position a permanent magnet closer to the region of action than the drive means. The drive means will tend to induce eddy currents in or on the selection means, leading to increased power consumption, reduced thermal stability and reduced achievable drive field strength. According to the present invention, the eddy currents induced in the selection means are completely or at least greatly reduced by virtue of providing at least one permanent magnet comprising a high resistive permanent magnet material.

According to the present invention, it is to be understood that the selection means and/or the drive means and/or the receiving means can at least partially be provided in the form of one single coil or solenoid. However, it is preferred according to the present invention that separate coils are provided to form the selection means, the drive means and the receiving means. Furthermore, the selection means can comprise a further permanent magnet located more distant from the region of action than the drive means. Furthermore according to the present invention, the selection means and/or the drive means and/or the receiving means can each be composed of separate individual parts, especially separate individual coils or solenoids, provided and/or arranged such that the separate parts form together the selection means and/or the drive means and/or the receiving means. Especially for the drive means and/or the selection means, a plurality of parts, especially pairs for coils (e.g. in a Helmholtz or Anti-Helmholtz configuration) are preferred in order to provide the possibility to generate and/or to detect components of magnetic fields directed in different spatial directions.

According to the present invention, it is preferred that the permanent magnet material is formed of blocks or parts which are small compared to the skin depth the of permanent magnet material for frequencies used for varying the magnetic drive field. Furthermore, it is preferred that blocks or parts of the permanent magnet material are electrically insulated from each other. According to the present invention, it is thereby possible to greatly reduce the strength of the eddy currents induced inside the at least one permanent magnet.

It is furthermore preferred according to the present invention that the permanent magnet is cooled by means of outside cooling means and/or by means of internal cooling means. By outside cooling means, it is to be understood according to the present invention that a cooling is applied to the permanent magnet from the outside surface of the permanent magnet. By internal cooling means, it is to be understood according to the present invention that a cooling is applied through the material of the permanent magnet.

One preferred example of internal cooling means is the realization of the cooling of the permanent magnet by means of cooling channels. Thereby, it is advantageously possible to easily define the temperature of the permanent magnet and to efficiently provide a heat transfer from the permanent magnet to the cooling medium.

According to the present invention, it is preferred that the arrangement is usable for influencing and/or detecting the magnetic particles in the region of action both together with the permanent magnet and without the permanent magnet. This advantageously allows for a very flexible use of the inventive arrangement as the permanent magnet can be used optionally, e.g. in order to locally enhance the spatial resolution power of the arrangement according to the present invention.

Furthermore, it is preferred that the permanent magnet is provided movable to different locations inside or outside of the region of action. This also allows for an enhanced flexibility in the use of the inventive arrangement.

In a further preferred embodiment of the present invention, the permanent magnet is located closer to the region of action compared to the location of the drive means or compared to at least parts of the drive means. Thereby, it is possible to with the arrangement according to the present invention to realize a comparably high magnetic field strength of the magnetic selection field and therefore a very steep gradient of the magnetic selection field in the area of the region of action. This in turn allows for a very high spatial resolution of the arrangement according to the present invention.

According to a further preferred embodiment of the present invention, the permanent magnet material is barium strontium ferrite or a so-called bonded magnet material. These materials both provide a relatively low electrical conductivity and the possibility to effectively prevent the generation of eddy currents inside the permanent magnet material. The so-called bonded magnets comprise a powdered material providing the magnetic characteristics and a, usually organic, binder material providing the mechanical characteristics. The powdered material is, e.g., provided such that the electrical conductivity is very much reduced (due to the fact that the grains of the powdered material do not or only slightly contact each other, e.g. by means of providing an electrical insulation by the binder material) relative to the electrical conductivity that the powdered material would have if it was not powdered but shaped in a solid block. If this reduction in electrical conductivity is not sufficient, it is preferred according to the present invention, to subdivide the bonded magnet material into blocks or parts which are small compared to the skin depth the of permanent magnet material for frequencies used for varying the magnetic drive field. The bonded magnet material can, e.g., be a metallic material.

According to the present invention, it is very advantageous to take into consideration a change in conducting properties of selection means or drive means if these means are penetrated by the magnetic field of each other. The resistance of the coil components of the selection means or the drive means should be chosen as low as possible in the given environment or penetration pattern and the electrical conductivity of the permanent magnet material penetrated by the magnetic drive fields should be chosen as low as possible. The selection means and the drive means together are also called “field generator means”. The selection means comprise magnetic field generation means that provide either a static (gradient) magnetic selection field and/or a comparably slowly changing long range magnetic selection field with frequencies in the range of about 1 Hz to about 100 Hz. Both the static part and the comparably slowly changing part of the magnetic selection field can be generated by means of permanent magnets or by means of coils or by a combination thereof. The drive means comprise magnetic field generation means that provide a magnetic drive field with frequencies in the range of about 1 kHz to about 200 kHz, preferably about 10 kHz to about 100 kHz.

The present invention further refers to the use of high resistive permanent magnet material in an arrangement according to the present invention and further to a method for influencing and/or detecting magnetic particles in a region of action, wherein the method comprises the steps of generating a magnetic selection field having a pattern in space of its magnetic field strength such that a first sub-zone having a low magnetic field strength and a second sub-zone having a higher magnetic field strength are formed in the region of action, changing the position in space of the two sub-zones in the region of action by means of a magnetic drive field so that the magnetization of the magnetic particles changes locally, wherein the generation of the magnetic selection field is performed at least partially by means of a permanent magnet comprising a high resistive permanent magnet material. This advantageously allows for a very high magnetic field strength in a region very close to or inside the region of action without a very complex overall setup of the inventive arrangement.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

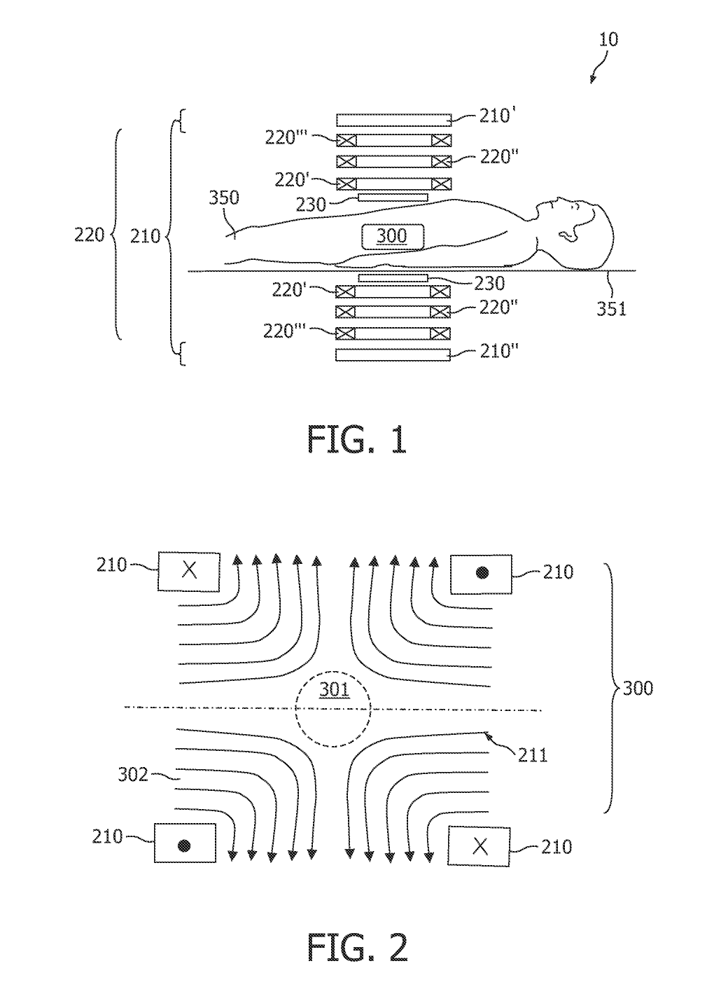

FIG. 1 illustrates an arrangement according to the present invention for carrying out the method according to the present invention.

FIG. 2 illustrates an example of the field line pattern produced by an arrangement according to the present invention

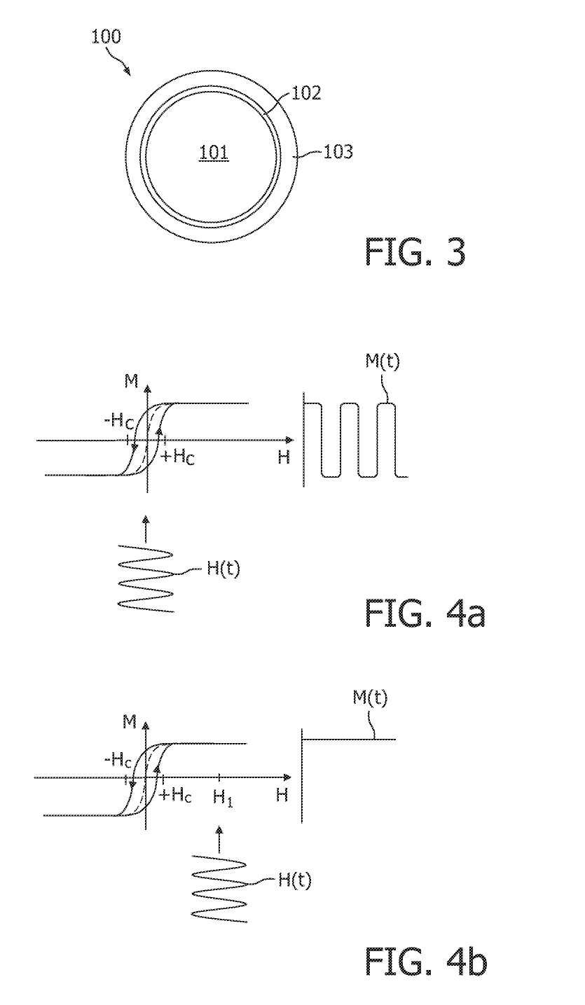

FIG. 3 illustrates an enlarged view of a magnetic particle present in the region of action.

FIGS. 4a and 4b illustrate the magnetization characteristics of such particles.

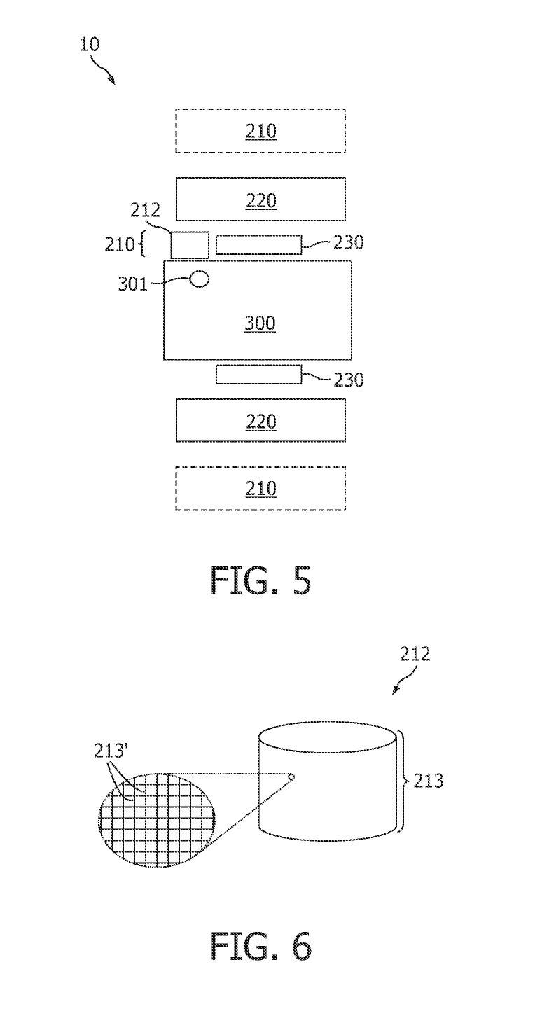

FIGS. 5 and 6 illustrate schematically different views of a permanent magnet.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described of illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIG. 1, an arbitrary object to be examined by means of an arrangement 10 according to the present invention is shown. The reference numeral 350 in FIG. 1 denotes an object, in this case a human or animal patient, who is arranged on a patient table, only part of the top of which is shown. Prior to the application of the method according to the present invention, magnetic particles 100 (not shown in FIG. 1) are arranged in a region of action 300 of the inventive arrangement 10. Especially prior to a therapeutical and/or diagnostical treatment of, for example, a tumor, the magnetic particles 100 are positioned in the region of action 300, e.g. by means of a liquid (not shown) comprising the magnetic particles 100 which is injected into the body of the patient 350.

As an example of an embodiment of the present invention, an arrangement 10 is shown in FIG. 2 comprising a plurality of coils forming a selection means 210 whose range defines the region of action 300 which is also called the region of treatment 300. For example, the selection means 210 is arranged above and below the patient 350 or above and below the table top. For example, the selection means 210 comprise a first pair of coils 210′, 210″, each comprising two identically constructed windings 210′ and 210″ which are arranged coaxially above and below the patient 350 and which are traversed by equal currents, especially in opposed directions. The first coil pair 210′, 210″ together are called selection means 210 in the following. Preferably, direct currents are used in this case.

According to the present invention, the selection means 210 comprise a permanent magnet which is only shown in FIGS. 5 and 6 and is referenced by reference sign 212. According to a preferred embodiment of the present invention, the permanent magnet 212 is optional.

The selection means 210 generate a magnetic selection field 211 which is in general a gradient magnetic field which is represented schematically in FIG. 2 by the field lines. It has a substantially constant gradient in the direction of the (e.g. vertical) axis of the coil pair of the selection means 210 and reaches the value zero in a point on this axis. Starting from this field-free point (not individually shown in FIG. 2), the field strength of the magnetic selection field 211 increases in all three spatial directions as the distance increases from the field-free point. In a first sub-zone 301 or region 301 which is denoted by a dashed line around the field-free point the field strength is so small that the magnetization of particles 100 present in that first sub-zone 301 is not saturated, whereas the magnetization of particles 100 present in a second sub-zone 302 (outside the region 301) is in a state of saturation. The field-free point or first sub-zone 301 of the region of action 300 is preferably a spatially coherent area; it may also be a punctiform area or else a line or a flat area. In the second sub-zone 302 (i.e. in the residual part of the region of action 300 outside of the first sub-zone 301) the magnetic field strength is sufficiently strong to keep the particles 100 in a state of saturation. By changing the position of the two sub-zones 301, 302 within the region of action 300, the (overall) magnetization in the region of action 300 changes. By measuring the magnetization in the region of action 300 or a physical parameters influenced by the magnetization, information about the spatial distribution of the magnetic particles in the region of action can be obtained. In order to change the relative spatial position of the two sub-zones 301, 302 in the region of action 300, a further magnetic field, the so-called magnetic drive field 221, is superposed to the selection field 211 in the region of action 300 or at least in a part of the region of action 300.

FIG. 3 shows an example of a magnetic particle 100 of the kind used together with an arrangement 10 of the present invention. It comprises for example a spherical substrate 101, for example, of glass which is provided with a soft-magnetic layer 102 which has a thickness of, for example, 5 nm and consists, for example, of an iron-nickel alloy (for example, Permalloy). This layer may be covered, for example, by means of a coating layer 103 which protects the particle 100 against chemically and/or physically aggressive environments, e.g. acids. The magnetic field strength of the magnetic selection field 211 required for the saturation of the magnetization of such particles 100 is dependent on various parameters, e.g. the diameter of the particles 100, the used magnetic material for the magnetic layer 102 and other parameters.

In the case of e.g. a diameter of 10 μm, a magnetic field of approximately 800 A/m (corresponding approximately to a flux density of 1 mT) is then required, whereas in the case of a diameter of 100 μm a magnetic field of 80 A/m suffices. Even smaller values are obtained when a coating 102 of a material having a lower saturation magnetization is chosen or when the thickness of the layer 102 is reduced.

For further details of the preferred magnetic particles 100, the corresponding parts of DE 10151778 are hereby incorporated by reference, especially paragraphs 16 to 20 and paragraphs 57 to 61 of EP 1304542 A2 claiming the priority of DE 10151778.

The size of the first sub-zone 301 is dependent on the one hand on the strength of the gradient of the magnetic selection field 211 and on the other hand on the field strength of the magnetic field required for saturation. For a sufficient saturation of the magnetic particles 100 at a magnetic field strength of 80 A/m and a gradient (in a given space direction) of the field strength of the magnetic selection field 211 amounting to 160 10³ A/m2, the first sub-zone 301 in which the magnetization of the particles 100 is not saturated has dimensions of about 1 mm (in the given space direction). By increasing the magnetic field strength and especially the magnetic gradient strength of the magnetic selection field 211, is its possible to enhance the spatial resolution of the arrangement 10 according to the present invention.

When a further magnetic field—in the following called a magnetic drive field 221 is superposed on the magnetic selection field 210 (or gradient magnetic field 210) in the region of action 300, the first sub-zone 301 is shifted relative to the second sub-zone 302 in the direction of this magnetic drive field 221; the extent of this shift increases as the strength of the magnetic drive field 221 increases. When the superposed magnetic drive field 221 is variable in time, the position of the first sub-zone 301 varies accordingly in time and in space. It is advantageous to receive or to detect signals from the magnetic particles 100 located in the first sub-zone 301 in another frequency band (shifted to higher frequencies) than the frequency band of the magnetic drive field 221 variations. This is possible because frequency components of higher harmonics of the magnetic drive field 221 frequency occur due to a change in magnetization of the magnetic particles 100 in the region of action 300 as a result of the non-linearity of the magnetization characteristics.

In order to generate these magnetic drive fields 221 for any given direction in space, there are provided three further coil pairs, namely a second coil pair 220′, a third coil pair 220″ and a fourth coil pair 220′ which together are called drive means 220 in the following. For example, the second coil pair 220′ generates a component of the magnetic drive field 221 which extends in the direction of the coil axis of the first coil pair 210′, 210″ or the selection means 210, i.e. for example vertically. To this end the windings of the second coil pair 220′ are traversed by equal currents in the same direction. The effect that can be achieved by means of the second coil pair 220′ can in principle also be achieved by the superposition of currents in the same direction on the opposed, equal currents in the first coil pair 210′, 210″, so that the current decreases in one coil and increases in the other coil. However, and especially for the purpose of a signal interpretation with a higher signal to noise ratio, it may be advantageous when the temporally constant (or quasi constant) selection field 211 (also called gradient magnetic field) and the temporally variable vertical magnetic drive field are generated by separate coil pairs of the selection means 210 and of the drive means 220.

The two further coil pairs 220″, 220″′ are provided in order to generate components of the magnetic drive field 221 which extend in a different direction in space, e.g. horizontally in the longitudinal direction of the region of action 300 (or the patient 350) and in a direction perpendicular thereto. If third and fourth coil pairs 220″, 220″′ of the Helmholtz type (like the coil pairs for the selection means 210 and the drive means 220) were used for this purpose, these coil pairs would have to be arranged to the left and the right of the region of treatment or in front of and behind this region, respectively. This would affect the accessibility of the region of action 300 or the region of treatment 300. Therefore, the third and/or fourth magnetic coil pairs or coils 220″, 220″′ are also arranged above and below the region of action 300 and, therefore, their winding configuration must be different from that of the second coil pair 220′. Coils of this kind, however, are known from the field of magnetic resonance apparatus with open magnets (open MRI) in which an radio frequency (RF) coil pair is situated above and below the region of treatment, said RF coil pair being capable of generating a horizontal, temporally variable magnetic field. Therefore, the construction of such coils need not be further elaborated herein.

The arrangement 10 according to the present invention further comprise receiving means 230 that are only schematically shown in FIG. 1. The receiving means 230 usually comprise coils that are able to detect the signals induced by magnetization pattern of the magnetic particles 100 in the region of action 300. Coils of this kind, however, are known from the field of magnetic resonance apparatus in which e.g. a radio frequency (RF) coil pair is situated around the region of action 300 in order to have a signal to noise ratio as high as possible. Therefore, the construction of such coils need not be further elaborated herein.

In an alternative embodiment for the selection means 210 shown in FIG. 1, further permanent magnets (not shown) can be used to generate the gradient magnetic selection field 211 in addition to the permanent magnet 212 comprising the low conductivity permanent magnet material according to the present invention. It is also possible according to the present invention to provide also the further permanent magnets with a low conductivity in order to suppress as much as possible the generation of eddy currents also in the further permanent magnets. In the space between two poles of such (opposing) further permanent magnets (not shown) there is formed a magnetic field which is similar to that of FIG. 2, that is, when the opposing poles have the same polarity. In another alternative embodiment of the arrangement according to the present invention, the selection means 210 comprise both at least one further permanent magnet and at least one coil 210′, 210″ as depicted in FIG. 2.

The frequency ranges usually used for or in the different components of the selection means 210, drive means 220 and receiving means 230 are roughly as follows: The magnetic field generated by the selection means 210 does either not vary at all over the time or the variation is comparably slow, preferably between approximately 1 Hz and approximately 100 Hz. The magnetic field generated by the drive means 220 varies preferably between approximately 25 kHz and approximately 100 kHz. The magnetic field variations that the receiving means are supposed to be sensitive are preferably in a frequency range of approximately 50 kHz to approximately 10 MHz.

FIGS. 4a and 4b show the magnetization characteristic, that is, the variation of the magnetization M of a particle 100 (not shown in FIGS. 4a and 4b) as a function of the field strength H at the location of that particle 100, in a dispersion with such particles. It appears that the magnetization M no longer changes beyond a field strength +H_{c} and below a field strength −H_{c}, which means that a saturated magnetization is reached. The magnetization M is not saturated between the values +H_{c} and −H_{c}.

FIG. 4a illustrates the effect of a sinusoidal magnetic field H(t) at the location of the particle 100 where the absolute values of the resulting sinusoidal magnetic field H(t) (i.e. “seen by the particle 100”) are lower than the magnetic field strength required to magnetically saturate the particle 100, i.e. in the case where no further magnetic field is active. The magnetization of the particle 100 or particles 100 for this condition reciprocates between its saturation values at the rhythm of the frequency of the magnetic field H(t). The resultant variation in time of the magnetization is denoted by the reference M(t) on the right hand side of FIG. 4a. It appears that the magnetization also changes periodically and that the magnetization of such a particle is periodically reversed.

The dashed part of the line at the centre of the curve denotes the approximate mean variation of the magnetization M(t) as a function of the field strength of the sinusoidal magnetic field H(t). As a deviation from this centre line, the magnetization extends slightly to the right when the magnetic field H increases from −H_{c} to +H_{c} and slightly to the left when the magnetic field H decreases from +H_{c} to −H_{c}. This known effect is called a hysteresis effect which underlies a mechanism for the generation of heat. The hysteresis surface area which is formed between the paths of the curve and whose shape and size are dependent on the material, is a measure for the generation of heat upon variation of the magnetization.

FIG. 4b shows the effect of a sinusoidal magnetic field H(t) on which a static magnetic field H₁ is superposed. Because the magnetization is in the saturated state, it is practically not influenced by the sinusoidal magnetic field H(t). The magnetization M(t) remains constant in time at this area. Consequently, the magnetic field H(t) does not cause a change of the state of the magnetization.

One important object according to the present invention is to provide an inventive arrangement such that a permanent magnet 212 is as much as possible transparent for the magnetic drive field 221 of the drive means 220. It is proposed according to the present invention to provide at least one such permanent magnet 212 with a comparably low electrical conductivity. Such a permanent magnet 212 is depicted in FIG. 6. FIG. 5 shows the overall setup of an inventive arrangement 10 with a permanent magnet 212 according to the present invention.

In FIG. 5, the selection means 210, the drive means 220 and the receiving means 230 are depicted schematically in relation to schematical representation of the region of action 300 and first sub-zone 301 (containing the field-free point). Usually, in such an arrangement 10, the selection means 210 are positioned, at least partially, further away from the region of action 300 than the drive means 220. According to the present invention, it is possible that the permanent magnet 212 as a part of the selection means 210 is positioned closer to the region of action 300 than the drive means 220. According to an alternative embodiment, it is even possible to provide the selection means 210 exclusively by means of the permanent magnet 212 (i.e. the parts of the selection means 210 represented in dashed lines in FIG. 5 are omitted).

In FIG. 6, one example of the permanent magnet 212 is represented, comprising a permanent magnet material 213. The permanent magnet material 213 is preferably provided in the form of small sub-blocks 213′ forming the desired geometry. These blocks 213′ are preferably electrically insulated against each other to avoid large loops for the eddy currents to flow. The size of the sub-blocks 213′ should be lower than the skin depth of the material at the frequency of the variation of the magnetic drive field. Usually, this means that the size of the sub-blocks 213′ should be smaller than one millimetres or a few millimetres. Preferably, the permanent magnet material 213 is formed such that cooling channels (not depicted) are provided inside the permanent magnet material 213.

1. An arrangement (10) for influencing and/or detecting magnetic particles (100) in a region of action (300), which arrangement comprises:
selection means (210) for generating a magnetic selection field (211) having a pattern in space of its magnetic field strength such that a first sub-zone (301) having a low magnetic field strength and a second sub-zone (302) having a higher magnetic field strength are formed in the region of action (300),
drive means (220) for changing the position in space of the two sub-zones (301, 302) in the region of action (300) by means of a magnetic drive field (221) so that the magnetization of the magnetic particles (100) changes locally,
wherein the selection means (210) comprise at least one permanent magnet (212) comprising a high resistive permanent magnet material (213).

2. An arrangement (10) according to claim 1, wherein the permanent magnet material (213) is formed of blocks or parts (213′) which are small compared to the skin depth the of permanent magnet material (213) for frequencies used for varying the magnetic drive field.

3. An arrangement (10) according to claim 2, wherein blocks or parts (213′) of the permanent magnet material (213) are electrically insulated from each other.

4. An arrangement (10) according to claim 1, wherein the permanent magnet (213) is cooled by means of outside cooling means and/or by means of internal cooling means.

5. An arrangement (10) according to claim 4, wherein the permanent magnet (212) comprises cooling channels.

6. An arrangement (10) according to claim 1, wherein the arrangement (10) is usable for influencing and/or detecting the magnetic particles (100) in the region of action (300) both together with the permanent magnet (212) and without the permanent magnet (212).

7. An arrangement (10) according to claim 1, wherein the permanent magnet (212) is provided movable to different locations inside or outside of the region of action.

8. An arrangement (10) according to claim 1, wherein the permanent magnet (212) is located closer to the region of action (300) compared to the location of the drive means (220) or compared to at least parts off the drive means (220).

9. An arrangement (10) according to claim 1, wherein the permanent magnet material (213) is barium strontium ferrite or a bonded magnet material (213).

10. A method for influencing and/or detecting magnetic particles (100) in a region of action (300), wherein the method comprises the steps of
generating a magnetic selection field (211) having a pattern in space of its magnetic field strength such that a first sub-zone (301) having a low magnetic field strength and a second sub-zone (302) having a higher magnetic field strength are formed in the region of action (300),
changing the position in space of the two sub-zones (301, 302) in the region of action (300) by means of a magnetic drive field (221) so that the magnetization of the magnetic particles (100) changes locally,
wherein the generation of the magnetic selection field (211) is performed at least partially by means of a permanent magnet (212) comprising a high resistive permanent magnet material (213).

11. The use of a high resistive permanent magnet material (213) in an arrangement (10) according to claim 1 for influencing and/or detecting magnetic particles (100) in a region of action (300).
